(19) United States
(12) Patent Application Publication
(10) Pub. No.: US 2023/0240816 A1
(43) Pub. Date: Aug. 3, 2023

(54) CARRIER TO FACILITATE ISQ MEASUREMENTS

(71) Applicant: Robert E. Chianelli, Montoursville, PA (US)

(72) Inventor: Robert E. Chianelli, Montoursville, PA (US)

(21) Appl. No.: 18/103,529

(22) Filed: Jan. 31, 2023

Related U.S. Application Data
(60) Provisional application No. 63/305,385, filed on Feb. 1, 2022.

(51) Int. Cl. A61C 8/00

(52) U.S. Cl. CPC A61C 8/0089

(57) ABSTRACT

A carrier tool is proposed that may be used in combination with a dental torque wrench to easily access all dental implants, regardless of their location within the mount. The carrier tool comprises a shaft for extending the reach of the wrench, with a shortened mount attached to an opposing end of the shaft. The shorted mount is used to securely hold the implant measurement device during insertion within the implant. A magnet is disposed within the interior of the mount to secure the implant measurement device (also magnetized) in place as the carrier tool is brought into the proper position with respect to the implant.

### CROSS-REFERENCE TO RELATED APPLICATIONS

This application claims priority from U.S. Provisional Application No. 63/305,385, filed Feb. 1, 2022 and incorporated herein by reference.

### TECHNICAL FIELD

The present invention relates to the field of dental instrumentation and, more particularly, to tool for improving the ability to access dental implants in posterior locations when performing osseointegration measurements.

### BACKGROUND OF THE INVENTION

Resonance frequency analysis (RFA) is widely used to determine the stability of a dental implant. This technique is favored over the reverse torque technique due to the fact it is non-invasive. RFA is a non-destructive quantitative measurement of implant integration (osseointegration) by assessing changes in implant stability over time. The technology consists of temporarily inserting a transducer post into the threaded opening at the implant surface and using a probe to generate magnetic pulses in proximity to the transducer post. The transducer post will begin to vibrate at a pulse frequency that creates a resonance between the post and probe output. The probe is configured to translate the resonant frequency an Implant Stability Quotient (ISQ) value. The higher the resonant frequency, the greater the stability of the implant. The RFA process may be used several times over a period of weeks to monitor the degree of osseointegration that is taking place.

FIG. 1 is a simplified drawing showing a typical transducer post 1 as inserted into an implant 2. An example transducer post 1 is shown in FIG. 2, including a threaded lower portion 3 that is particularly manufactured to match the thread pitch in the central opening of implant 2. A magnetized component 4 is included at the opposing end of post 1. Once detector bar 1 is screwed down into position, a probe 5 (as shown in FIG. 1) is brought into a defined position with respect to transducer post 1 and is energized to transmit magnetic pulses of increasing frequency toward the post.

While an extremely useful tool, the ability to properly insert the transducer post within implants located in certain posterior areas is problematic and, if not properly seated, may result in errors in the ISQ measurements. The number of implants, in combination with various angles results in many instances where there is limited access for performing ISQ measurements, especially in the posterior area of the mouth. This is observed most notably in the maxillary arch and in cases where the implants are tipped mesailly to avoid the sinus cavity. In some situations, an implant may be angled by as much as 30 degrees. Even single implant cases in the posterior or second molar areas have limited access to the implant platform when the remaining dentition is present.

### SUMMARY OF THE INVENTION

As described in detail below, the present invention is directed to the field of dental instrumentation and, more particularly, to a tool for improving the ability to access dental implants in posterior locations when performing osseointegration measurements.

In accordance with the present invention, a carrier tool is proposed that may be used in combination with a dental torque wrench to easily access all dental implants, regardless of their location within the mount. The carrier tool comprises a shaft for extending the reach of the wrench, with a shortened mount (used for holding the implant measurement device during positioning) attached to an opposing end of the shaft. As with prior art mounting arrangements, a magnet is disposed within the interior of the mount to secure the implant measurement device (also magnetized) securely in place as brought into the proper position.

An exemplary embodiment of the present invention may take the form of an apparatus for seating a transducer post in a dental implant for an ISQ measurement. The apparatus (which may also be referred to as a “carrier tool”) comprising: a shaft including a fitting on an upper termination (the fitting configured to provide attachment to a head of a right-angle dental torque wrench), and a transducer post mount coupled to a lower end termination of the shaft. The transducer post mount includes a central opening for supporting a magnetic element at a location for attracting an associated transducer post. The internal cross-section of the transducer post mount configured to mate with a torquing feature of the transducer post and provide for proper insertion of the transducer post within the dental implant as required for a resonant frequency measurement.

Other and further embodiments and aspects of the present invention will become apparent during the course of the following discussion and by reference to the accompanying drawings.

### BRIEF DESCRIPTION OF THE DRAWINGS

Referring now to the drawings, where like numerals represent like parts in different views:

FIG. 1 is a depiction of a typical arrangement used to perform resonance frequency analysis of a dental implant, used to provide a measurement of the implant stability quotient (ISQ);

FIG. 2 is a depiction of an example transducer post that is temporarily screwed into a dental implant, the vibration of the transducer post indicative of the resonant frequency associated with the implant;

FIG. 3 is a cut-away view of an exemplary carrier tool formed in accordance with the present invention and used to ensure the proper placement and seating of a transducer post within a dental implant;

FIG. 4 is an outlined isometric view of the carrier tool of FIG. 3;

FIG. 5 is an isometric, solid rendering of the carrier tool of FIGS. 3 and 4;

FIG. 6 illustrates an example of a placement of the inventive carrier tool with respect to a dental wrench used for attaching the transducer post to an implant; and

FIG. 7 illustrates a typical dental polishing cup tool, with the cup itself removed to illustrate the ability to use the remaining element of the tool as the shaft portion of the inventive carrier tool.

### DETAILED DESCRIPTION

Implant stability quotient (ISQ) measurements can be obtained using commercially available instruments and detectors placed on the implant. With an increased number of patients seeking dental implant treatment, there has been a significant increase in the number of full arch multi-implant restorations provided. A large percentage of full arch implant cases may require several individual implants placed at various convergent and divergent angles. The various orientations of these implants (as well as their location within the mouth, as discussed below) may create problems in terms of the ability to properly access the implant and orient the temporary transducer post so that it will seat properly within the implant and generate an accurate ISQ measurement.

This is concern most notably in the maxillary arch and in cases where the implants are tipped mesailly to avoid the sinus cavity. In some situations, an implant may be angled by as much as 30 degrees. Even single implant cases in the posterior or second molar areas have limited access to the implant platform when the remaining dentition is present. The difficulty in accessing these areas may easily result in the transducer post not being properly engaged with the implant, where mis-adjustments may result in incorrect resonance values (which may go unnoticed by the practitioner).

The present invention addresses the access problem for ISQ measurements and discloses an inventive carrier element that is able to adjust the angular presentation of a transducer post with respect to the implant location and ensure a proper seating of the post, providing an increased confidence in the ISQ measurement results.

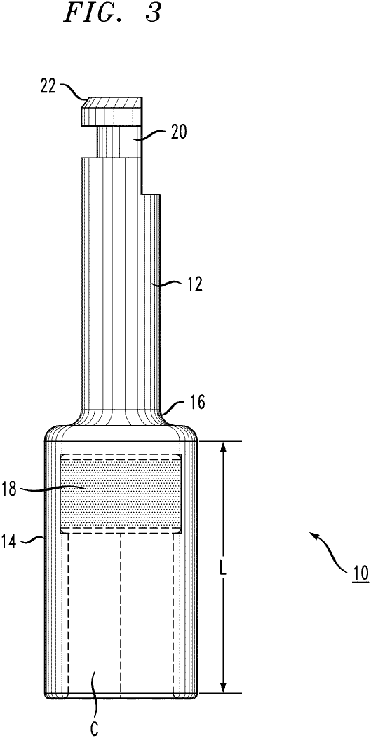

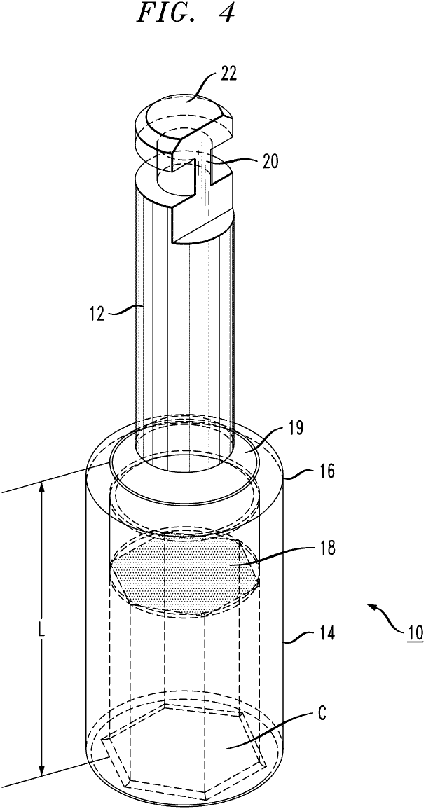

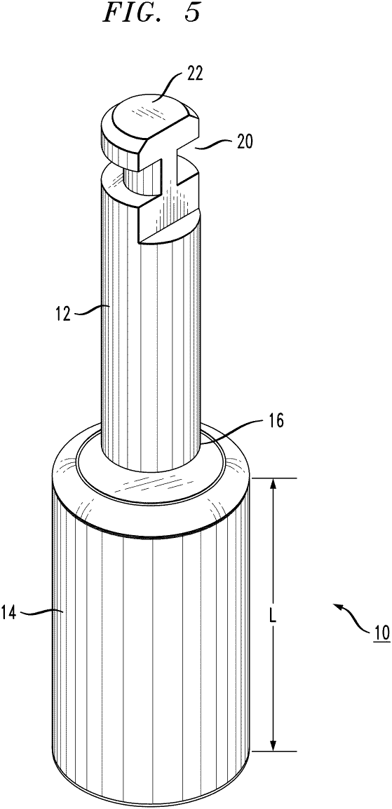

FIG. 3 is a cut-away side view of a carrier tool formed in accordance with the present invention, with FIG. 4 showing a cut-away isometric view and FIG. 5 presenting a “solid” rendition of the same carrier tool. Referring to FIGS. 3-5, a carrier tool 10 is shown as comprising a shaft component 12 and an associated transducer post mount 14, with shaft component 12 attached to a top end termination 16 of mount 14. In accordance with the principles of the present invention, the length L of mount 14 is sized to be relatively short when compared to conventional mounts used for ISQ measurements, allowing for easier access to a mouth with limited vertical height.

In describing the attachment of shaft 12 to mount 14, it is contemplated that various arrangements may be used for this purpose. For example, a central aperture 19 (see FIG. 4) may be formed in top end termination 16, central aperture 19 having a diameter only slightly greater than the diameter of shaft 12 to allow for a secure fit. Indeed, a threaded attachment between these components of carrier tool 10 may be used. Shaft 12 may be releasably attached to mount 14 (such as by using the threaded attachment) or permanently bonded to mount 14 by using a proper adhesive, solder compound, or welding operation (for example). Moreover, it is contemplated that carrier tool 10 may be formed as a single piece, as best shown in the rendering of FIG. 5. This unitary configuration may be formed by a machining or injection molding, for example.

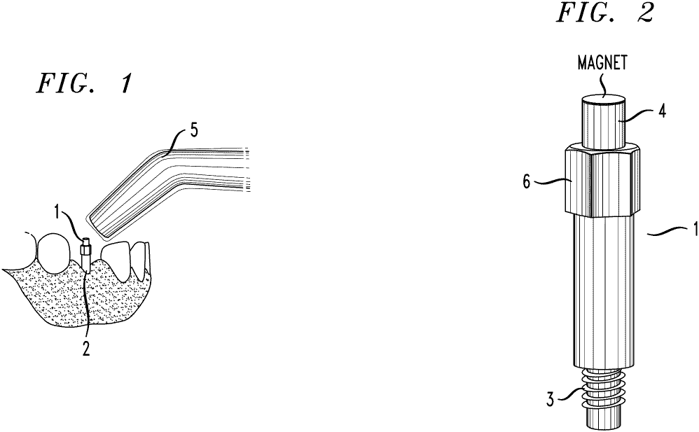

As with the prior art mounts used for this purposes, mount 14 has a hollow central region C for capturing the distal termination of a transducer post. The internal cross-section of region C is configured to mate with a torquing element of the post (an example torque element 6 shown on the post of FIG. 2). A magnet 18 is disposed within hollow central region C, where its relative position with respect to mount 14 is clearly shown in FIG. 3. Magnet 18 is used to attract magnetized component 4 of an exemplary post 1 (see FIG. 2). The coupling of magnet 18 and magnetized component 4 is used to hold the transducer post in place during the transit of carrier tool 10 from an instrument table to the patient’s implant site.

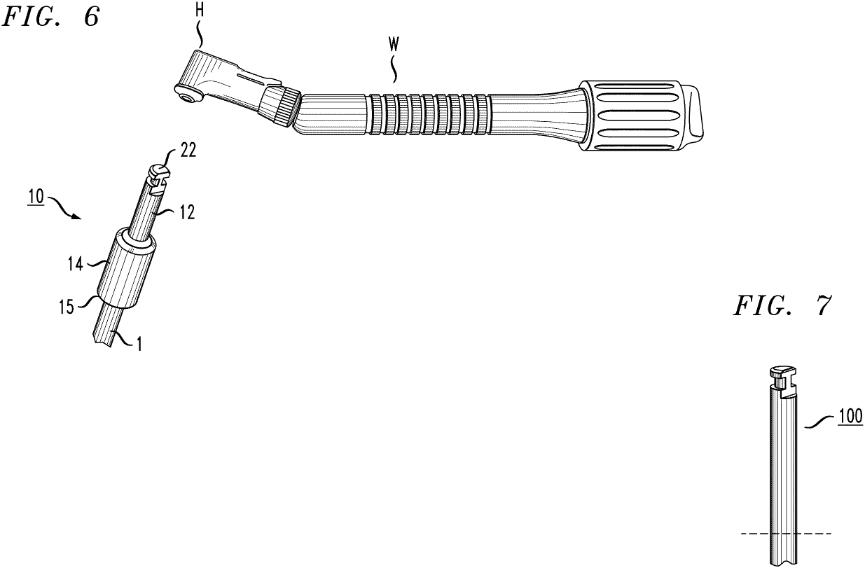

Continuing with reference to FIGS. 3 - 5, a distal end 20 of shaft component 12 is shown as comprising a fitting 22 that is configured to mate with a working end of a dental torque wrench. FIG. 6 illustrates in diagrammatic form (and not to scale) carrier tool 10 and a dental torque wrench W, showing the presentation of fitting 22 of carrier tool 10 with respect to the right-angled head H of wrench W. Thus, once carrier tool 10 is engaged with wrench W, a transducer post 1 may be inserted into the bottom opening of mount 14 and held in place while its torquing element 6 is rotated by the combination of wrench W and carrier tool 10 until post 1 is properly seated within the implant. In accordance with the principles of the present invention, the addition of carrier tool 10 enabling the proper presentation of transducer post 1 to limited access positions (including convergent/divergent angular displacements) of dental implants.

Advantageously, carrier tool 10 may be easily and quickly assembled from a general dentist’s armamentarium. For example, shaft component 12 may be created by using a polishing cup, with the cup portion removed to expose a fitting useful in combination with a wrench. FIG. 7 illustrates an exemplary adaptation of a conventional polishing cup 100. A conventional mount used for ISQ measurements may be used for mount 14, where the conventional mount is sectioned to reduce its length. When sectioning, it is critical to ensure that magnet 18 remains in its original location.

It is to be understood that the scope of the present invention is not limited to the above-described specific embodiments and description, and it will be appreciated by those skilled in the art that changes may be made in this embodiments without departing from the principles and spirit of the invention, the scope of which is defined by the claims appended hereto.

What is claimed is:

1. Apparatus for seating a transducer post in a dental implant for an ISQ measurement, the apparatus comprising:
a shaft including a fitting on an upper termination, the fitting configured to provide attachment to a head of a right-angle dental torque wrench; and
a transducer post mount coupled to a lower end termination of the shaft, the detector mount including an embedded magnetic element, the transducer mount including a central opening for supporting a magnetic element at a location for attracting an associated transducer post, an internal cross-section of the transducer post mount configured to mate with a torquing feature of the transducer post and provide for proper insertion of the transducer post within the dental implant as required for a resonant frequency measurement.

2. The apparatus as defined in claim 1, wherein
The transducer post mount further comprises an aperture formed in the upper termination thereof, the aperture having a diameter only slightly greater that the diameter of the shaft to provide for a coupling of the shaft with the transducer post mount in an aligned configuration.

3. The apparatus as defined in claim 2, wherein
The shaft includes a threaded portion along a portion of the lower termination; and
The aperture within the transducer post mount comprises a threaded aperture for mating with the threaded portion of the shaft.

4. The apparatus as defined in claim 2, wherein the shaft is press-fit into the aperture of the shaft.

5. The apparatus as defined in claim 1, wherein the lower termination of the shaft is adhesively bonded to an upper termination of the transducer post mount.

6. The apparatus as defined in claim 1, wherein the lower termination of the shaft is mechanically welded to an upper termination of the transducer post mount.

7. The apparatus as defined in claim 1, wherein the lower termination of the shaft is soldered to an upper termination of the transducer post mount.

8. The apparatus as defined in claim 1, wherein the shaft and the transducer post mount are formed as a single, unitary component.

9. The apparatus as defined in claim 8 wherein the single, unitary component comprises an injection-molded component.
